(19) United States
(12) Patent Application Publication
(10) Pub. No.: US 2016/0045470 A1
(43) Pub. Date: Feb. 18, 2016

(54) AMORPHOUS SOLID DISPERSION OF TREPROSTINIL DIETHANOLAMINE

(71) Applicant: Dr. Reddy's Laboratories Limited, Hyderabad (IN)

(72) Inventors: Peddireddy Subba Reddy, Hyderabad (IN); Jithin K. Jose, Kottayam (Dist) (IN)

(21) Appl. No.: 14/824,865

(22) Filed: Aug. 12, 2015

(30) Foreign Application Priority Data
Aug. 12, 2014 (IN) 3957/CHE/2014

(51) Int. Cl. A61K 31/205; A61K 31/133; A61K 9/10

(52) U.S. Cl. CPC A61K 31/205; A61K 9/10; A61K 31/133

(57) ABSTRACT

The present application relates to the amorphous solid dispersion of treprostinil diethanolamine salt and the process for its preparation.

This patent application claims priority to Indian Provisional Patent Application number 3957/CHE/2014 filed on Aug. 12, 2014, the contents of which are incorporated by reference herein.

### BACKGROUND

The present application relates to the amorphous solid dispersion of treprostinil diethanolamine salt and the process for its preparation.

The drug compound having the adopted name treprostinil diethanolamine, has a chemical name acetic acid, 2-[[(1R,2R,3aS,9aS)-2,3,3a,4,9,9a-hexahydro-2-hydroxy-1-[(3S)-3-hydroxyoctyl]-1H-benz[f]inden-5-yl]oxy]-, complexed with 2,2′ -iminobis[ethanol], and is represented by structure of formula I.

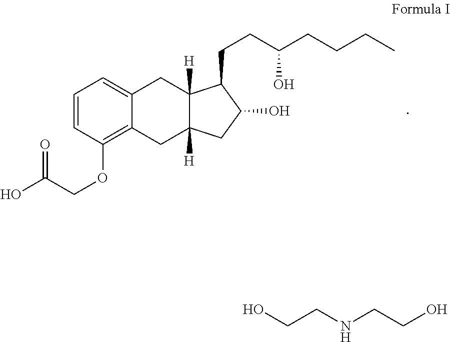 [SMILES: CCCC[C@H](O)CC[C@@H]1[C@H]2Cc3cccc(OCC(=O)O)c3C[C@H]2C[C@H]1O ; OCCNCCO]

Treprostinil diethanolamine is a prostacyclin vasodilator indicated for treatment of pulmonary arterial hypertension (PAH) (WHO Group 1) to improve exercise capacity. Process for preparation of treprostinil, treprostinil derivatives and intermediates useful in preparation of treprostinil are described in U.S. Pat. Nos. 4,306,075; 6,700,025; 6,809,223 and 6,765,117.

U.S. Pat. No. 7,417,070 discloses crystalline Form A, crystalline Form B, mixture of crystalline Form A & B and amorphous form of treprostinil diethanolamine salt and processes for preparation thereof.

It has been disclosed earlier that the amorphous forms in a number of drugs exhibit different dissolution characteristics and in some cases different bioavailability patterns compared to crystalline forms [Konne T., Chem pharm Bull., 38, 2003(1990)]. For some therapeutic indications one bioavailabihty pattern may be favoured over another. An amorphous form of Cefuroxime axetil is a good example for exhibiting higher bioavailability than the crystalline form.

There remains a need to provide stable, commercially viable and advantageous solid state form of treprostinil diethanolamine.

### SUMMARY

In the first embodiment, the present application provides a solid dispersion comprising an amorphous form of treprostinil diethanolamine and one or more pharmaceutically acceptable carriers.

In the second embodiment, the present application provides a solid dispersion comprising an amorphous form of treprostinil diethanolamine and one or more pharmaceutically acceptable carriers characterized by powder X-ray diffraction (PXRD) substantially as illustrated by FIG. 1 or FIG. 2.

In the third embodiment, the present application provides a process for preparing a solid dispersion comprising an amorphous form of treprostinil diethanolamine and one or more pharmaceutically acceptable carriers, which comprises;
a) providing a solution comprising treprostinil diethanolamine and one or more pharmaceutically acceptable carriers in a solvent,
b) removing solvent from a solution obtained in step (a), and
c) recovering a solid dispersion comprising an amorphous form of treprostinil diethanolamine and one or more pharmaceutically acceptable carriers.

### BRIEF DESCRIPTION OF THE DRAWINGS

FIG. 1 is powder X-ray power diffraction (“PXRD”) pattern of a solid dispersion comprising an amorphous form of treprostinil diethanolamine and PVP-K30 prepared according to Example 1.

FIG. 2 is powder X-ray power diffraction (“PXRD”) pattern of a solid dispersion comprising an amorphous form of treprostinil diethanolamine and hydroxypropyl methylcellulose acetate succinate (HPMC-AS) prepared according to Example 2.

### DETAILED DESCRIPTION

In the first embodiment, the present application provides a solid dispersion comprising an amorphous form of treprostinil diethanolamine and one or more pharmaceutically acceptable carriers.

Solid dispersion as used herein refers to the dispersion of one or more active ingredients in an inert excipient or matrix (carrier), where the active ingredients could exist in finely crystalline, solubilized or amorphous state (Sareen et al., 2012 and Kapoor et al., 2012). Solid dispersion consists of two or more than two components, generally a carrier polymer and drug optionally along with stabilizing agent (and/or surfactant or other additives). The most important role of the added polymer in solid dispersion is to reduce the molecular mobility of the drug to avoid the phase separation and re-crystallization of drug during storage. The increase in solubility of the drug in solid dispersion is mainly because drug remains in amorphous form which is associated with a higher energy state as compared to crystalline counterpart and due to that it required very less external energy to dissolve.

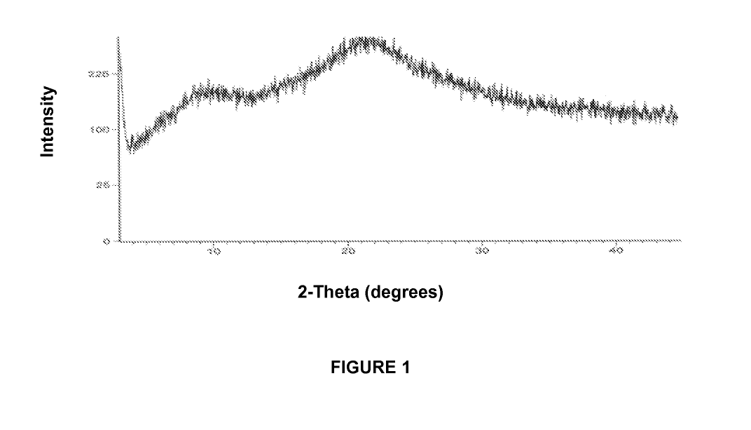

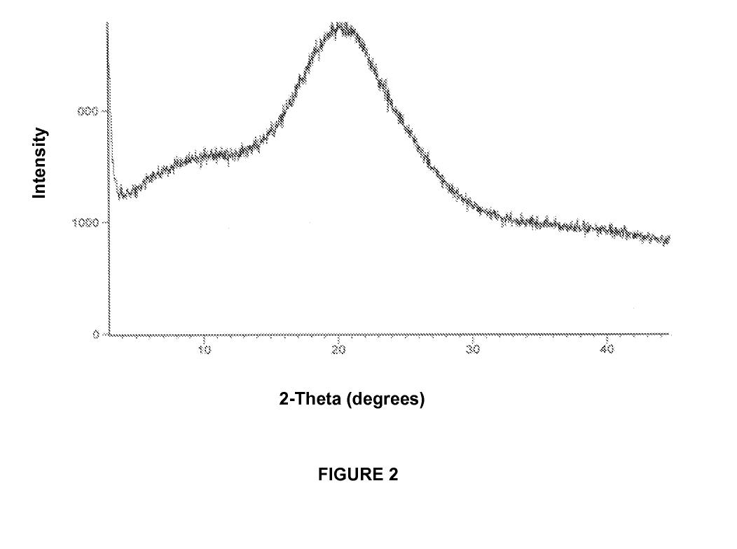

In the second embodiment, the present application provides a solid dispersion comprising an amorphous form of treprostinil diethanolamine and one or more pharmaceutically acceptable carriers characterized by powder X-ray diffraction (PXRD) substantially as illustrated by FIG. 1 or FIG. 2.

In the third embodiment, the present application provides a process for preparing a solid dispersion comprising an amorphous form of treprostinil diethanolamine and one or more pharmaceutically acceptable carriers, which comprises;
a) providing a solution comprising treprostinil diethanolamine and one or more pharmaceutically acceptable carriers in a solvent,
b) removing solvent from a solution obtained in step (a), and
c) recovering a solid dispersion comprising an amorphous form of treprostinil diethanolamine and one or more pharmaceutically acceptable carriers.
   Providing a solution in step a) includes:
i) direct use of a reaction mixture containing treprostinil diethanolamine that is obtained in the course of its synthesis and adding one or more pharmaceutically acceptable carriers; or
ii) direct use of a reaction mixture containing treprostinil diethanolamine that is obtained by treating treprostinil with diethanolamine and adding one or more pharmaceutically acceptable carriers; or
iii) dissolving treprostinil, diethanolamine and one or more pharmaceutically acceptable carriers in a solvent; or
iv) dissolving treprostinil diethanolamine in a solvent and adding one or more pharmaceutically acceptable carriers.

Any physical form of treprostinil diethanolamine may be utilized for providing the solution of treprostinil diethanolamine in step (a).

Suitable pharmaceutically acceptable carriers which can be used in step (a) include, but are not limited to: diluents such as starches, pregelatinized starches, lactose, powdered celluloses, microcrystalline celluloses, dicalcium phosphate, tricalcium phosphate, mannitol, sorbitol, xylitol, sugar and the like; binders such as acacia, guar gum, tragacanth, gelatin, polyvinylpyrrolidones such as polyvinylpyrrolidones(PVP K-30,K-90), poly (vinyl pyrrolidone-co-vinyl acetate) (PVP-VA) and the like, hydroxypropyl celluloses, hydroxypropyl methylcellulose, cellulose acetate, hydroxypropyl methylcellulose acetate succinate (HPMC-AS) and the like; disintegrants such as starches, sodium starch glycolate, pregelatinized starches, crospovidones, croscarmellose sodium and the like; lubricants such as stearic acid, magnesium stearate, zinc stearate and the like; glidants such as colloidal silicon dioxide and the like; solubility or wetting enhancers such as anionic or cationic or neutral surfactants; maltodextrin, complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants, and the like.

Suitable solvents which can be used for dissolving the diethanolamine salt of treprostinil include but are not limited to: alcoholic solvents such as methanol, ethanol, isopropyl alcohol, n-propanol, isoamyl alcohol and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, di-isopropyl ether, 1,4-dioxane and the like; hydrocarbons such as toluene, xylene and the like; nitriles such as acetonitrile, propionitrile and the like; and any mixtures of two or more thereof.

After dissolution in step (a), optionally undissolved particles, if any, may be removed suitably by filtration, centrifugation, decantation, and any other known techniques. The solution can be filtered by passing through paper, glass fiber, or other membrane material, or a clarifying agent such as celite. Depending upon the equipment used and the concentration and temperature of the solution, the filtration apparatus may need to be preheated to avoid premature crystallization.

Step (b) involves removing solvent from a solution obtained in step (a);

Suitable techniques which can be used for the removal of solvent include but not limited to evaporation, flash evaporation, simple evaporation, rotational drying, spray drying, agitated thin-film drying, agitated nutsche filter drying, pressure nutsche filter drying, freeze-drying or any other technique known in the art.

Step (c) involves recovering a solid dispersion comprising an amorphous form of treprostinil diethanolamine and one or more pharmaceutically acceptable carriers. The said recovery can be by using the processes known in the art.

The resulting compound obtained in step (c) may be optionally further dried. Drying can be carried out in a tray dryer, vacuum oven, air oven, cone vacuum dryer, rotary vacuum dryer, fluidized bed dryer, spin flash dryer, flash dryer, or the like. The drying can be carried out at temperatures of less than about 60° C., less than about 50° C., less than about 40° C., less than about 30° C., less than about 20° C., or any other suitable temperatures; at atmospheric pressure or under a reduced pressure; as long as the treprostinil diethanolamine is not degraded in its quality. The drying can be carried out for any desired times until the required product quality is achieved. Suitable time for drying can vary from few minutes to several hours for example from about 30 minutes to about 24 or more hours.

The solid dispersion of the present invention is thus suitable for the preparation of a pharmaceutical composition, in particular a solid pharmaceutical composition such as a solid oral dosage form, like a tablet, powders, capsules, suppositories, sachets, transdermal patch and the like.

Treprostinil or its diethanolamine salt which may be used as the input in the process for preparation of the solid states of the present application can be prepared by any process known in the art.

Solid states of treprostinil diethanolamine of the present application are characterized by its PXRD pattern. All PXRD data reported herein were obtained using Cu Kα radiation, having the wavelength 1.541 A, and were obtained using a PanAlytical, Powder X-ray Diffractometer.

Although the exemplified procedures herein illustrate the practice of the present invention in some of its embodiments, the procedures should not be construed as limiting the scope of the invention. Modifications from consideration of the specification and examples within the ambit of current scientific knowledge will be apparent to one skilled in the art.

The following definitions are used in connection with the present application unless the context indicates otherwise.

“Amorphous form” as used herein refers to a solid state wherein the amorphous content within the said solid state is at least about 35% or at least about 40% or at least about 45% or at least about 50% or at least about 55% or at least about 60% or at least about 65% or at least about 70% or at least about 75% or at least about 80% or at least about 85% or at least about 90% or at least about 95% or at least about 96% or at least about 97% or at least about 98% or at least about 99% or about 100%.

An “alcohol” is an organic compound containing a carbon bound to a hydroxyl group. “C1-C6 alcohols” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, isoamyl alcohol, diethylene glycol monomethyl ether, diethylene glycol monoethyl ether, cyclohexanol, phenol, glycerol, or the like.

An “aliphatic hydrocarbon” is a liquid hydrocarbon compound, which may be linear, branched, or cyclic and may be saturated or have as many as two double bonds. A liquid hydrocarbon compound that contains a six-carbon group having three double bonds in a ring is called “aromatic.” Examples of “C5-C8 aliphatic or aromatic hydrocarbons” include, but are not limited to, isopentane, neopentane, isohexane, 3-methylpentane, 2,3-dimethylbutane, neohexane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, methylcyclohexane, cycloheptane, petroleum ethers, benzene toluene, ethylbenzene, m-xylene, o-xylene, p-xylene, trimethylbenzene, chlorobenzene, fluorobenzene, trifluorotoluene, anisole, or any mixtures thereof.

An “ester” is an organic compound containing a carboxyl group —(C═O))—O— bonded to two other carbon atoms. “C3-C6 esters” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, or the like.

An “ether” is an organic compound containing an oxygen atom —O— bonded to two other carbon atoms. “C2-C6 ethers” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, or the like.

A “halogenated hydrocarbon” is an organic compound containing a carbon bound to a halogen. Halogenated hydrocarbons include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone” is an organic compound containing a carbonyl group —(C═O)— bonded to two other carbon atoms. “C3-C6 ketones” include, but are not limited to, acetone, ethyl methyl ketone, diethyl ketone, methyl isobutyl ketone, ketones, or the like.

A “nitrile” is an organic compound containing a cyano —(C≡N) bonded to another carbon atom. “C2-C6 Nitriles” include, but are not limited to, acetonitrile, propionitrile, butanenitrile, or the like.

All percentages and ratios used herein are by weight of the total composition and all measurements made are at about 25° C. and about atmospheric pressure, unless otherwise designated. All temperatures are in degrees Celsius unless specified otherwise. As used herein, “comprising” means the elements recited, or their equivalents in structure or function, plus any other element or elements which are not recited. The terms “having” and “including” are also to be construed as open ended. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

Example 1: Preparation of a solid dispersion comprising an amorphous form of treprostinil diethanolamine and PVP-K30.

Treprostinil diethanolamine (300 mg) and PVP-K30 (300 mg) was dissolved in methanol (8 mL) and stirred for 10 min at 28° C. The resultant reaction mass solvent was completely evaporated under reduced pressure at 59° C. Separated solid was dried to afford title compound.

Example 2: Preparation of a solid dispersion comprising an amorphous form of treprostinil diethanolamine and hydroxy propyl methyl cellulose (HPMC-AS).

Treprostinil diethanolamine (300 mg) and HPMC-AS (300 mg) was dissolved in methanol (34 mL) and stirred for 10 min at 28° C. The resultant reaction mass solvent was completely evaporated under reduced pressure at 59° C. Separated solid was dried to afford title compound.

Comparative example: Treprostinil diethanolamine (400 mg) was dissolved in mixture of ethanol (2.1 mL) & water (2.1 mL) and stirred for 20 min at 28° C. The resultant reaction mass solvent was completely evaporated by employing fast evaporation technique. The obtained product was a viscous liquid.

We claim:

1. A solid dispersion comprising an amorphous form of treprostinil diethanolamine and one or more pharmaceutically acceptable carriers.

2. The solid dispersion according to claim 1, characterized by powder X-ray diffraction (PXRD) substantially as illustrated by FIG. 1 or FIG. 2.

3. The solid dispersion according to claim 1, wherein the pharmaceutically acceptable carriers are polyvinylpyrrolidones, poly (vinyl pyrrolidone-co-vinyl acetate) (PVP-VA), hydroxypropyl celluloses, hydroxypropyl methylcellulose, cellulose acetate, hydroxypropyl methylcellulose acetate succinate (HPMC-AS) or mixture thereof.

4. The solid dispersion according to claim 1, wherein the pharmaceutically acceptable carriers are polyvinylpyrrolidones, cellulose acetate, hydroxypropyl methylcellulose acetate succinate (HPMC-AS) or mixture thereof.

5. Process for the preparation of the solid dispersion comprising an amorphous form of treprostinil diethanolamine and one or more pharmaceutically acceptable carriers, which comprises;
(a) providing a solution comprising treprostinil diethanolamine and one or more pharmaceutically acceptable carriers in a solvent,
(b) removing solvent from a solution obtained in step (a), and
(c) recovering a solid dispersion comprising an amorphous form of treprostinil diethanolamine and one or more pharmaceutically acceptable carriers.

6. The process according to claim 5, wherein the solvent in step (a) is selected from methanol, ethanol, isopropyl alcohol, n-propanol, isoamyl alcohol, acetone, ethyl methyl ketone, methyl isobutyl ketone, ethyl acetate, n-propyl acetate, n-butyl acetate, t-butyl acetate, toluene, acetonitrile or mixtures thereof.

7. The process according to claim 6, where in the solvent is selected from methanol, ethanol, isopropyl alcohol, acetone and mixtures thereof.

8. The process according to claim 5, wherein the removal of solvent in step (b) is carried out by evaporation using rotational drying or spray drying or agitated thin-film drying.

9. A pharmaceutical composition comprising an amorphous form of treprostinil diethanolamine and a pharmaceutically acceptable carrier.

10. A pharmaceutical composition according to claim 10, wherein composition is in the form of tablet or powders or capsules or suppositories or sachets or transdermal patch
